(19) United States
(12) Patent Application Publication
(10) Pub. No.: US 2026/0069397 A1
(43) Pub. Date: Mar. 12, 2026

(54) MANAGING DEBRIS-PROTECTION IN RELATION TO VASCULAR MEDICAL PROCEDURES OR MEDICAL PROCEDURES TO A HEART

(71) Applicant: Zeev BRANDEIS, Rosh HaAyian (IL)

(72) Inventor: Zeev BRANDEIS, Rosh HaAyian (IL)

(21) Appl. No.: 19/106,267

(22) PCT Filed: Aug. 24, 2023
(86) PCT No.: PCT/IL2023/050902

§ 371 Date: Feb. 25, 2025

Related U.S. Application Data
(60) Provisional application No. 63/400,779, filed on Aug. 25, 2022.
(60) Provisional application No. 63/400,773, filed on Aug. 25, 2022.
(60) Provisional application No. 63/400,771, filed on Aug. 25, 2022.

(51) Int. Cl. A61F 2/01

(52) U.S. Cl. CPC A61F 2/01; A61F 2002/016; A61F 2250/0004

(57) ABSTRACT

A method of managing debris-protection associated with medical procedures, the method including, activating a debris-protection device placed in a body lumen to prevent debris from flowing with body fluids to a body organ prior to beginning a step in a medical procedure which is likely to produce debris, and de-activating the debris-protection device before the activated debris-protection device is likely to cause damage to the body organ. An activation mechanism for activating a debris-protection device associated with a medical procedure, the mechanism including a spring against which force must be exerted to activate the device. An activation mechanism for activating a debris-protection device associated with a medical procedure, wherein the mechanism is programmed to automatically activate the device based on an activation input. Related apparatus and methods are also described.

### RELATED APPLICATIONS

This application is a PCT application which claims the benefit of priority of U.S. Provisional Ser. No. 63/400,773 filed 25 Aug. 2022 titled “Managing Debris-Protection in Relation to Vascular Medical Procedures or Medical Procedures to a Heart”; of U.S. Provisional Ser. No. 63/400,771 filed 25 Aug. 2022, titled “PROTECTING CORONARY ARTERIES AND MYOCARDIUM BLOOD VESSELS DURING MEDICAL PROCEDURES”; of U.S. Provisional Ser. No. 63/400,779 filed 25 Aug. 2023, titled “FIXATION OF DEBRIS PROTECTION DEVICES IN BODY LUMENS”; and is related to U.S. Provisional Ser. No. 63/398,546 filed on Aug. 17, 2022, titled “CAPTURING FLOWING DEBRIS IN BLOOD VESSELS AND OTHER BODY LUMENS”.

The contents of all of the above applications are incorporated by reference as if fully set forth herein.

### FIELD AND BACKGROUND

The present disclosure, in some embodiments thereof, relates to methods and devices for managing debris-protection in relation to vascular medical procedures or medical procedures to a heart, and, more particularly, but not exclusively, to managing debris-protection in relation to procedure-related medical operations expected to produce blood-borne debris, that might injure and/or damage and/or block blood vessels in the brain and/or the heart and/or other body organs.

Background art includes:

International Patent Application Publication Number WO 2019/064223 of Brandeis, which describes an aortic protection device including a mesh lumen shaped and sized to extend along the aorta, from a heart-side of a brachiocephalic artery exit from the aorta to distal of a left subclavian artery exit from the aorta, wherein the mesh lumen is arranged to change a porosity of mesh pores in response to external control.

The disclosures of all references mentioned above and throughout the present specification, as well as the disclosures of all references mentioned in those references, are hereby incorporated herein by reference.

### SUMMARY

The present disclosure, in some embodiments thereof, relates to methods and devices for managing debris-protection in relation to vascular medical procedures or medical procedures to a heart, and, more particularly, but not exclusively, to managing debris-protection in relation to medical operations expected to produce blood-borne debris.

According to an aspect of some embodiments of the present disclosure there is provided a method of managing debris-protection associated with medical procedures, the method including activating a debris-protection device placed in a body lumen to prevent debris from flowing with body fluids to a body organ prior to beginning a step in a medical procedure which is likely to produce debris, and de-activating the debris-protection device before the activated debris-protection device is likely to cause damage to the body organ.

According to some embodiments of the disclosure, the activating includes activating a debris capture device placed in an aorta.

According to some embodiments of the disclosure, the activating includes activating a filtering of blood entering coronary arteries.

According to some embodiments of the disclosure, the activating includes blocking blood from entering coronary arteries.

According to some embodiments of the disclosure, the activating is performed prior to performing a pace-up step in a valve replacement medical procedure.

According to some embodiments of the disclosure, the activating is performed simultaneously to performing a pace-up step in a valve replacement medical procedure.

According to some embodiments of the disclosure, the activating comprises activating filtering blood through a mesh with a mesh pore size in a range from 120 to 500 microns.

According to some embodiments of the disclosure, the activating comprises activating prior to positioning an implantable device at a location at a heart.

According to some embodiments of the disclosure, the activating comprises activating filtering blood through a mesh with a mesh pore size in a range from 30 to 120 microns.

According to some embodiments of the disclosure, the activating comprises activating filtering blood through a mesh with a mesh pore size in a range from 0 to 6 microns.

According to some embodiments of the disclosure, wherein the activating comprises activating prior to expanding an implantable device at a location at a heart.

According to some embodiments of the disclosure, wherein the activating is performed prior to performing a pace-down step in a valve replacement medical procedure.

According to some embodiments of the disclosure, the de-activating is performed after a duration in a range from 5 seconds to 60 seconds.

According to some embodiments of the disclosure, the de-activating is performed after a duration in a range from 5 seconds to 120 seconds.

According to some embodiments of the disclosure, the de-activating is performed automatically by a timer.

According to some embodiments of the disclosure, the de-activating is performed automatically based on a signal from a sensor.

According to some embodiments of the disclosure, the activating is performed automatically based on a signal from a sensor.

According to some embodiments of the disclosure, the activating includes activating a debris capture device place in a Vena Cava.

According to some embodiments of the disclosure, the activating includes activating a debris filtering device place in a Vena Cava.

According to some embodiments of the disclosure, the activating requires exerting force.

According to an aspect of some embodiments of the present disclosure there is provided an activation mechanism for activating a debris-protection device associated with a medical procedure, the mechanism including a spring against which force must be exerted to activate the device.

According to some embodiments of the disclosure, further including automatic de-activation based on a de-activation input.

According to some embodiments of the disclosure, the de-activation input includes input from a timer set for a specific duration of activating the debris-protection device.

According to some embodiments of the disclosure, the de-activation input includes input from a pulse rate sensor.

According to some embodiments of the disclosure, the de-activation input includes input from a blood oxygen sensor.

According to an aspect of some embodiments of the present disclosure there is provided an activation mechanism for activating a debris-protection device associated with a medical procedure, wherein the mechanism is programmed to automatically activate the device based on an activation input.

According to an aspect of some embodiments of the present disclosure there is provided a user interface for managing a debris-protection device the user interface including an indication selected from a group consisting of an activation indication, a de-activation indication, a timer, a patient pulse rate indication, a debris-in-body-fluid indication, a blood oxygen indication, a particle indication based on intracranial Doppler, a particle indication based on B-wave Doppler, a particle indication based on spectral Doppler, a particle indication based on opto-fluidic sensors, and a particle indication based on an impedance sensor.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIG. 1A is a simplified line drawing illustration of a debris protection device such as described in above-mentioned International Patent Application Publication Number WO 2019/064223 of Brandeis;

FIG. 1B is a simplified line drawing illustration of a debris protection device such as described in above-mentioned U.S. Provisional Patent Application titled “Protecting Coronary Arteries and Myocardium Blood Vessels During Medical Procedures” (Attorney Docket Number 91186);

FIG. 1C is a simplified line drawing illustration of a debris protection device such as described in above-mentioned U.S. Provisional Patent Application titled “Protecting Coronary Arteries and Myocardium Blood Vessels During Medical Procedures” (Attorney Docket Number 91186);

FIG. 1D is a simplified illustration of a debris capture device such as described in above-mentioned U.S. Provisional Patent Application No. 63/398,546, located in an aorta according to an example embodiment;

FIG. 1E is a simplified illustration of a potential location for a debris trapping device such as described in above-mentioned U.S. Provisional Patent Application No. 63/398,546, located in a vein according to some example embodiments;

FIG. 2 is a simplified flow chart illustration of a method of performing a medical procedure protected by one or more debris protection devices according to an example embodiment; and

FIG. 3 is a simplified block diagram illustration of a method of performing a medical procedure protected by one or more debris protection devices according to an example embodiment.

### DESCRIPTION OF SPECIFIC EMBODIMENTS

The present disclosure, in some embodiments thereof, relates to methods and devices for managing debris-protection in relation to vascular medical procedures or medical procedures to a heart, and, more particularly, but not exclusively, to managing debris-protection in relation to medical operations expected to produce blood-borne debris.

### OVERVIEW

### Introduction

Various medical procedures are expected to produce debris, which may be borne by flow of body fluids to various body organs which may suffer damage from the debris. A patient undergoing such medical procedures may benefit from the medical procedure, only to subsequently suffer from debris damage.

Some non-limiting examples of medical procedures which are expected to produce blood-borne debris include: medical procedures to a heart, such as electrophysiology procedures, Patent Foramen Ovale (PFO) procedures, heart valve repairs, open heart surgery, percutaneous aortic valve replacement (PAVR), percutaneous aortic valve implantation (PAVI), transcatheter aortic valve implantation (TAVI), and transcatheter aortic valve replacement (TAVR). Such medical procedures are expected to produce debris which will flow downstream from a heart, and maybe reach coronary arteries, a brain, the kidneys, and so on.

Additional non-limiting examples of medical procedures which are expected to produce blood-borne debris include: medical procedures to a body which release debris into veins, which may then flow to the right side of the heart, and be pumped into the lungs, which may severely impact the lungs.

Debris may include organized and unorganized blood clots, calcification, tissue particles of various sources, non-body particles such as polymers and other materials, gaseous particles such as gas bubbles.

The gaseous particles as well as other types of debris can be introduced into vasculature via medical procedures, medical devices and equipment including heart lung machines, Extracorporeal membrane oxygenation (ECMO) machines, also known as extracorporeal life support (ECLS) machines, heart and vascular assist machines, and blood filtration equipment.

The debris may include large particles, such as 120 to 500 microns, and even bigger particles, such as 500 microns to 1000 microns and bigger, and smaller particles, sometimes called micro-emboli, in a size range of 30-60 microns and under.

Managing debris capture is intended to prevent blockage in arteries branching from the aorta which can injure the celiac trunk, the superior mesenteric arteries, the renal arteries, the gonadal artery, and the inferior mesenteric arteries.

In some embodiments, by way of a non-limiting example in a patient with pre-known diseases, malformations, and malfunctions-the debris capture and/or filtering may optionally be set to a small pore size, to prevent further damage to organs such as the intestinal duct, the pelvic organs, the kidneys, and the liver.

In some embodiments, for the above patient condition, debris capture duration may be changed so as not to exacerbate the pre-known condition, for example the duration can be prolonged to protect more, or shortened to prevent lack of blood.

### Some Non-limiting Examples of Debris Protection Devices

Some non-limiting example devices for debris protection include devices for filtering blood from entering blood vessels leading to debris-sensitive organs; devices for trapping debris flowing along blood vessels; and devices for blocking blood from entering blood vessels leading to debris-sensitive organs.

In some embodiments, filtering devices can include filtering through a larger-pore-size mesh (for example in a range of 120 microns to 500 microns), or filtering through a smaller-pore-size mesh (for example in a range of 30 microns to 120 microns), or filtering through a smallest-pore-size mesh (for example in a range of 6 microns to 30 microns), or even completely blocking blood flow by using a filter with pores sized 0-6 microns.

In some embodiments, the filtering device are optionally controllable to be activated, from not filtering, or from filtering with a larger-pore-size, to filtering with smaller or even smallest-pore size.

Some non-limiting example devices for debris protection include devices for trapping debris flowing along blood vessels.

In some embodiments, the filtering device are optionally controllable to be activated, from not trapping debris, or from trapping with traps having a larger-pore size, to filtering with smaller-pore size or even smallest-pore size.

Some non-limiting example devices for debris protection include devices for blocking blood from entering blood vessels leading to debris-sensitive organs.

In some embodiments, the blocking device may be placed at an entry to the blood vessels leading to the debris-sensitive organs.

In some embodiments, the blocking device may be a one-way obstacle or a one-wat valve, to prevent back flow of debris-laden blood. By way of a non-limiting example a one-way valve activated in an aorta may prevent blood-borne debris from flowing back into the coronary arteries during diastole.

In some embodiments, the blocking device is optionally controllable to be activated, from not blocking to blocking, and de-activated from blocking to not-blocking.

In some embodiments, a combination of filtering and blocking is optionally used. By way of a non-limiting example, a one-way valve may include mesh flaps, so that when activated, the one-way valve filters blood flowing back rather than completely blocking the blood from flowing back.

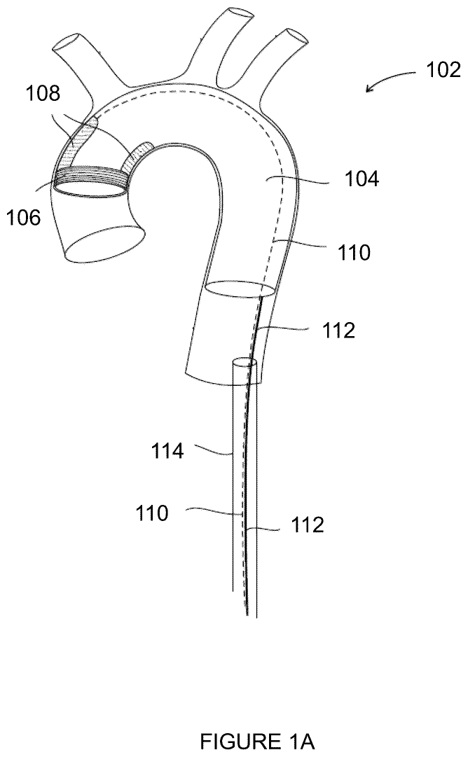

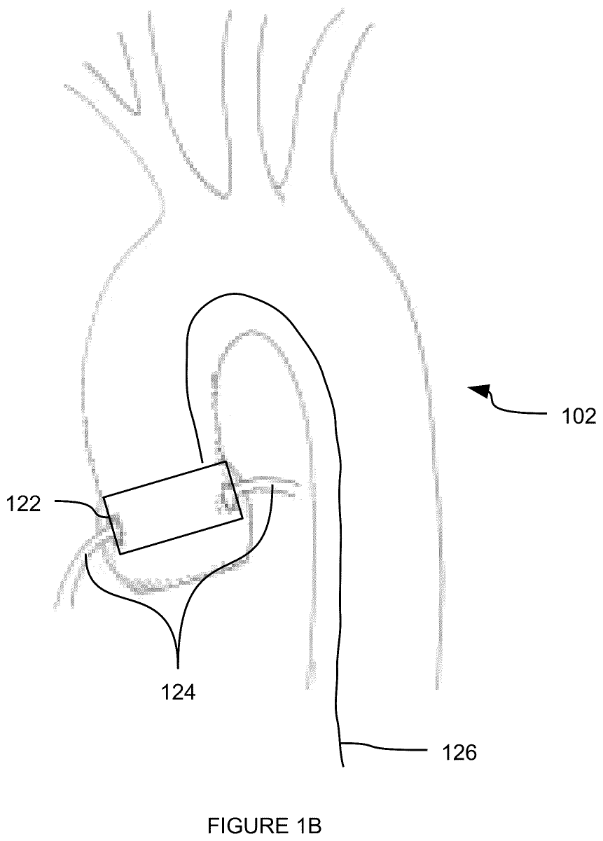

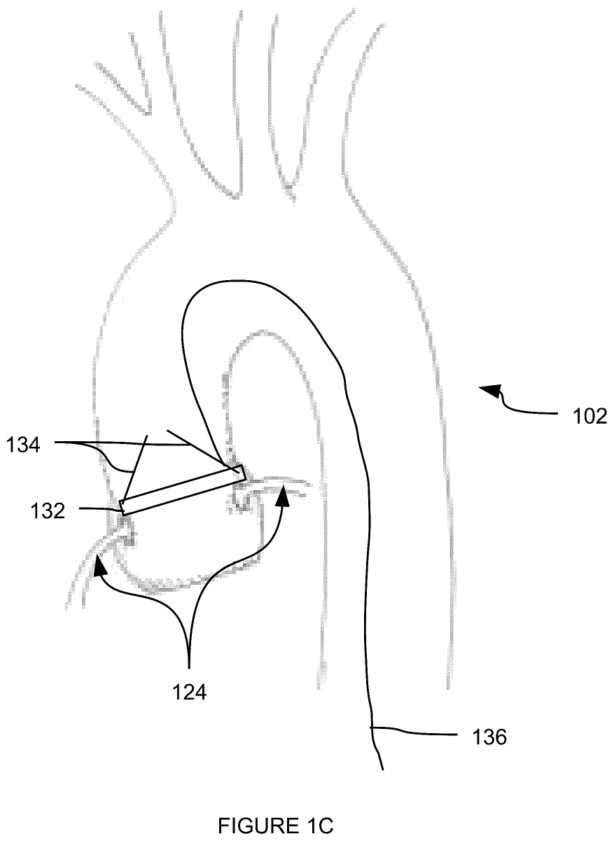

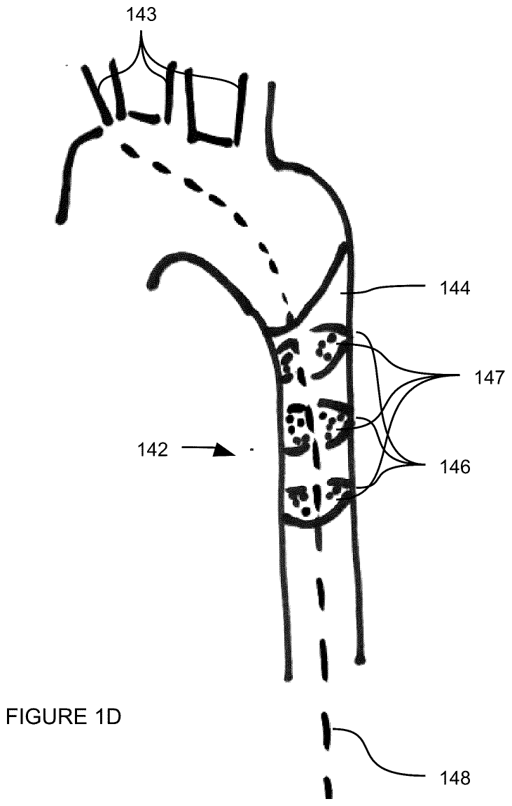

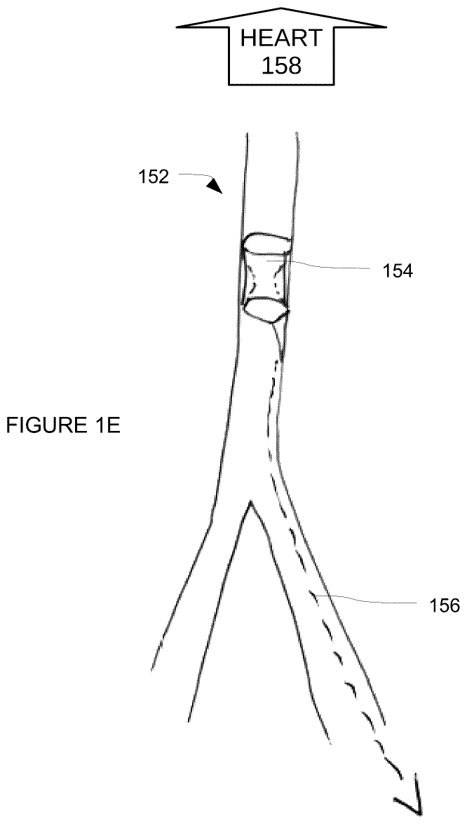

Some non-limiting examples of devices for debris protection are shown in FIGS. 1A to 1E.

### Duration of Activation

An aspect of some embodiments relates to managing duration of activation of devices for managing debris-protection.

In some embodiments, the managing may be blocking blood from entering coronary arteries.

By way of a non-limiting example, blocking entry of blood to coronary arteries, is optionally of a short duration, for example 5, 10, 20, 30 to 60 seconds. The heart is not deprived of blood for longer than the above-mentioned duration.

By way of a non-limiting example, blocking entry of blood to coronary arteries, is optionally equal to a duration of a Rapid Ventricular Pacing (RVP) step in a TAVI procedure, also called a “pace-up” step, where a patient's pulse is raised to a point of tachycardia-which provides little or no blood supply to the heart and brain.

By way of a non-limiting example, blocking entry of blood to coronary arteries, is optionally equal to a duration of a Rapid Ventricular Pacing (RVP) step in a TAVI procedure, also called a “pace-up” step, plus a short duration after, when the heart is expected to resume pumping effectively and a debris storm may be expected. In some embodiments, the example direction is equal to pace-up duration plus a short additional duration not to exceed 5, 10, 20, 30 to 60 seconds in total.

In some embodiments, the managing may be by filtering blood entering arteries.

In some embodiments, a mesh with relatively-large pores, for example pores sized approximately 120 microns, then the duration may be longer than in a case of complete blocking, for example a duration up to 1 minutes, 2 minutes, 5 minutes, and longer, potentially even without a time limit.

In some embodiments, a mesh with relatively-smaller pores, for example pores sized in a range between approximately 30 microns and approximately 120 microns, then the duration may be shorter than the above-mentioned filtering, and longer than the above-mentioned blocking duration, or even the same as the above-mentioned blocking duration. By way of a non-limiting example, the duration may optionally be in a range of 20 to 60 seconds.

In some embodiments, a mesh with relatively-smallest pores, for example pores sized smaller than 30 microns, then the duration may be even shorter than the above-mentioned filtering, and the same as the above-mentioned blocking duration.

In some embodiments, the managing may be blocking potentially-debris-laden blood from entering brain arteries.

The brain is typically able to withstand longer periods without blood than the heart muscle, and duration of activation of debris-protection is typically longer than the durations listed above with reference to debris protection for coronary arteries.

In some embodiments, the managing may be blocking potentially-debris-laden blood from entering arteries to kidneys. It is noted that a kidney can tolerate ischemia for a longer period than a brain or a heart.

### Timing of Activation

An aspect of some embodiments relates to managing timing of activation of devices for managing debris-protection.

In some embodiments, the managing may be blocking blood from entering coronary arteries or brain arteries. In such embodiments, the duration is expected to be relatively short, as described above, and the timing is optionally related to steps in a related medical procedure for which the debris protection is intended.

In some embodiments, the timing is different, depending on whether the blocking is to coronary arteries or brain arteries.

In some embodiments, the managing may be filtering blood. In such embodiments, the duration may be longer, as described above, and the timing may start even before, or together with, a beginning of a debris-producing step of the medical procedure.

In some embodiments, the activating is optionally before starting a medical procedure which is expected to produce debris.

In some embodiments, the activating is optionally before starting a step in a medical procedure which is expected to produce debris.

In some embodiments, the activating is before pace-up in a TAVI procedure.

In some embodiments, the activating is simultaneously with performing pace-up in a TAVI procedure.

In some embodiments, the activating is before pace-down in a TAVI procedure.

In some embodiments, the activating is simultaneously with performing pace-down in a TAVI procedure.

### Automatic Activation

An aspect of some embodiments relates to automatic activation of devices for managing debris-protection.

In some embodiments, activating devices for managing debris-protection is optionally based on data from a sensor.

In some embodiments, the sensor may optionally be a sensor such as, by way of a non-limiting example: a pulse rate sensor; a sensor for sensing debris by image analysis; a sensor for sensing activation by detecting radio-opaque markers attached to the devices for managing debris-protection; a blood oxygen sensor: a B wave ultrasound; an intracranial Doppler; a particle sensor in a heart, in an aorta, in an ascending aorta, in cerebral arteries, in a descending aorta, in the inferior Vena Cava and in the superior Vena Cava.

By way of a non-limiting example, a pulse rate sensor may indicate timing of a pace-up step, or indicate timing of a pace-down step.

By way of a non-limiting example, sensing debris by image analysis (for example of X-ray images produced during a medical procedure) may indicate presence of debris, and indicate that debris-protection is potentially desirable.

In some embodiments, the sensor may optionally provide an audible and/or a visible signal, and a physician may optionally activate and/or de-activate devices for managing debris-protection based on the signal.

In some embodiments, the sensor may optionally provide a signal which causes automatic de-activation of devices for managing debris-protection based on the signal. Such a feature is a potential safety feature.

In some embodiments, the sensor may optionally provide a signal which causes automatic activation of devices for managing debris-protection based on the signal.

### Safety features

An aspect of some embodiments relates to patient safety for operation of devices for managing debris-protection.

In some embodiments, the debris-protection device is a device with a normally-open state, normally not blocking or filtering fluid flow unless activated.

In some embodiments, the debris-protection device is a device with a normally-open state, normally not blocking fluid flow unless activated, but optionally filtering fluid flow, optionally using a pore size which does not damage organs which are downstream of the filtering.

In some embodiments, activating the debris-protection device is performed against resistance. For example, a physician manually performing the activation is optionally required to exert force to activate. In some embodiments, when exerting the force is stopped, the debris-protection device is automatically de-activated.

In some embodiments, the debris-protection device is automatically de-activated by a timer.

In some embodiments, a timer is automatically started when the debris-protection device is activated, and the timer activates a warning signal after a desired activation duration has passed.

### User Interface

An aspect of some embodiments relates to a user interface for operation of devices for managing debris-protection.

In some embodiments, the user interface optionally includes an indication of a state of a debris capture device. The indication optionally includes: activated; not activated; an indication of a pore size of a filter associated with the debris capture device; an indicate pf elapsed time since activation; a measure of an amount of detected debris; and a patient's pulse rate.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

Reference is now made to FIG. 1A, which is a simplified line drawing illustration of a debris protection device such as described in above-mentioned International Patent Application Publication Number WO 2019/064223 of Brandeis.

FIG. 1A shows an aortic protection device deployed in an aorta 102, the aortic protection device having two mesh layers—a first mesh layer 104 and a compactly packed second mesh layer 108.

FIG. 1A also shows an expandable anchoring stent 106 at a heart-proximal side of the aortic protection device, a first control wire 112 attached to a heart-distal end of the device, and a second control wire 110 attached to a heart-proximal end of the device and/or to the expandable anchoring stent 106, and a tube 114 through which the first control wire 112 and the second control wire 110 pass to outside a body of a patient.

Reference is now made to FIG. 1B, which is a simplified line drawing illustration of a debris protection device such as described in above-mentioned U.S. Provisional Patent Application titled “Protecting Coronary Arteries and Myocardium Blood Vessels During Medical Procedures” (Attorney Docket Number 91186).

FIG. 1B shows a simplified schematic block illustration of a coronary artery 124 protection device 122 deployed in an aorta 102, the coronary artery protection device connected to a control wire 126.

Reference is now made to FIG. 1C, which is a simplified line drawing illustration of a debris protection device such as described in above-mentioned U.S. Provisional Patent Application titled “Protecting Coronary Arteries and Myocardium Blood Vessels During Medical Procedures” (Attorney Docket Number 91186).

FIG. 1C shows a simplified schematic block illustration of a coronary artery 124 protection device 132 deployed in an aorta 102, the coronary artery protection device 124 including a one way valve 134, and the coronary artery protection device connected to a control wire 136. Reference is now made to FIG. 1D, which is a simplified illustration of a debris capture device such as described in above-mentioned U.S. Provisional Patent Application No. 63/398,546, located in an aorta according to an example embodiment.

FIG. 1D show a debris capture device 144, optionally having one or more levels of debris traps 146. The debris capture device 144 is optionally located in the descending aorta 142.

FIG. 1D also shows a medical tool 148, or a catheter 148, or a control wire 148 for a medical tool passing through the debris capture device 144, with the debris capture device 144 in an activated state, that is, debris traps are open, and the debris trapping leaves extend toward the middle of the descending aorta.

FIG. 1D shows debris particles 147 caught in the debris traps 146. When the debris traps 146 are optionally closed, the trapped debris 147 can optionally be extracted from the body together with the debris capture device 144.

Reference is now made to FIG. 1E which is a simplified illustration of a potential location for a debris trapping device such as described in above-mentioned U.S. Provisional Patent Application No. 63/398,546, located in a vein according to some example embodiments.

FIG. 1E shows the Vena Cava 152, and a debris trapping device 154 located in the Vena Cava 152. The Vena Cava 152 collects blood from veins to a right atrium of the heart 158. The heart pumps the blood to the lungs. If debris reaches the lungs it may be very detrimental.

In some embodiments, the debris trapping device 154 is placed in the Vena Cava 152, to block such debris.

In some embodiments, the debris trapping device 154 traps debris and is eventually withdrawn from a body, together with the debris.

In some embodiments, debris traps in the debris trapping device 154 are closed to contain trapped debris prior to withdrawing from the body together with the debris.

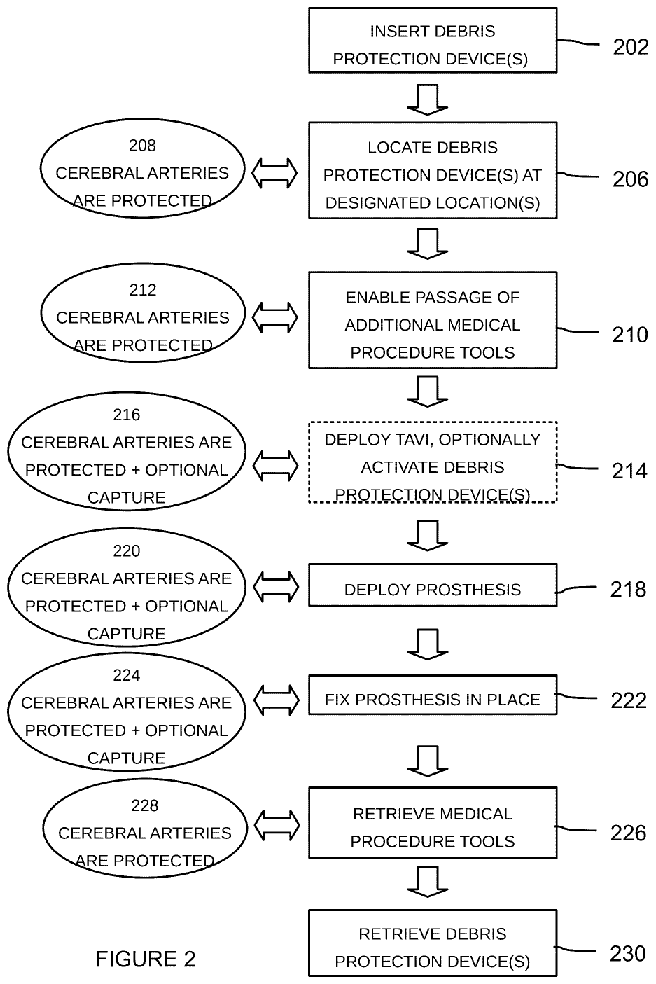

Reference is now made to FIG. 2, which is a simplified flow chart illustration of a method of performing a medical procedure protected by one or more debris protection devices according to an example embodiment.

FIG. 2 is intended to show steps in performing a TAVI procedure protected by one or more debris protection devices according to an example embodiment. By way of a non-limiting example, the debris protection devices may be as shown in FIGS. 1A-1D, and/or as described in above-mentioned U.S. Provisional Patent Application No. 63/398,546; co-filed U.S. Provisional Patent Application titled “Protecting Coronary Arteries and Myocardium Blood Vessels During Medical Procedures” (Attorney Docket Number 91186); U.S. Provisional Patent Application titled “Fixation of Debris Protection Devices in Body Lumens” (Attorney Docket 90786); and International Patent Application Publication Number WO 2019/064223 of Brandeis.

The method of FIG. 2 includes:
inserting one or more debris protection devices (202). The insertion may optionally be by femoral access which is either the same as the access for the TAVI procedure, or from a different access location, for example a different leg;
locating and/or deploying the one or more debris protection devices at their designated location(s) (206). From this time on, the cerebral arteries, for example, are protected (208);
enabling passage of additional medical procedure tools (210), for example TAVY tools and/or prosthesis. Enabling passage is achieved by having a passage open along the debris protection device. At this time, the cerebral arteries, for example, are protected (212);
deploying the TAVI prosthesis, and optionally activating debris protection device(s) (214), optionally providing a higher level of debris protection. For example, decreasing pore size of a filtering mesh, or activating a particle capture device. At this time, the cerebral arteries, for example, are protected (216), optionally even at a higher degree of protection, optionally by a low-permeability filter and/or particle capture device;
deploying the TAVI prosthesis (218). The deploying may optionally require repositioning. At this time, the cerebral arteries, for example, are protected (220), optionally EVEN by a low-permeability filter and/or particle capture device;
fixing the TAVI prosthesis in place (222). The fixing is performed by balloon inflation. At this time, the cerebral arteries, for example, are protected (220), optionally even by a low-permeability filter and/or particle capture device;
retrieving the TAVI medical tools (226). At this time, at least the cerebral arteries, for example, are protected (228); and
retrieving the debris protection device(s) (230).

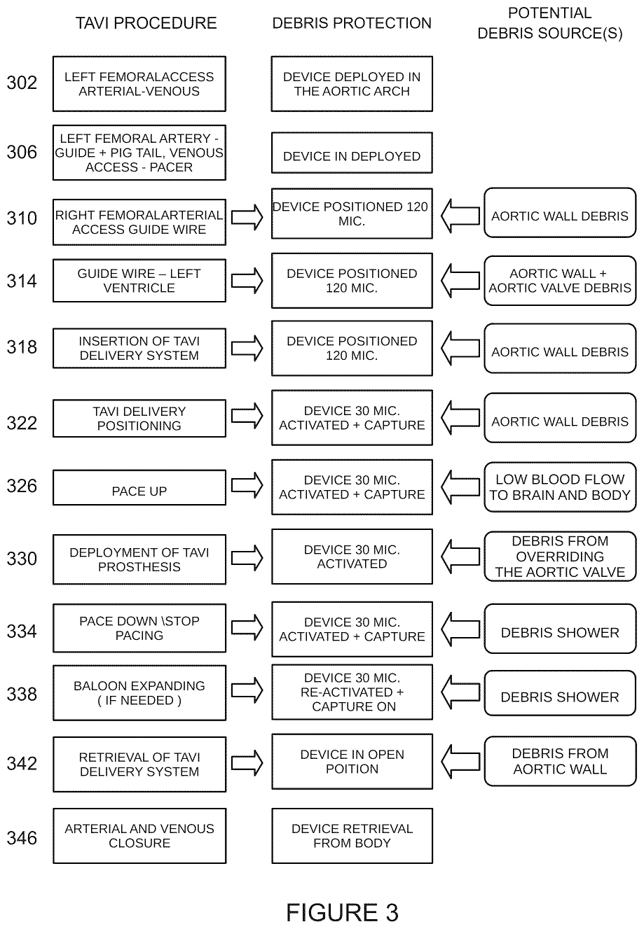

Reference is now made to FIG. 3, which is a simplified block diagram illustration of a method of performing a medical procedure protected by one or more debris protection devices according to an example embodiment.

FIG. 3 shows three columns of blocks. A left column shows steps in the TAVI procedure, a middle column shows corresponding steps in operating one or more debris protection device(s), and the right column shows potential debris sources at times corresponding to the left and middle column steps.

A first row 302 shows a step of obtaining left femoral arterial and venous access for the TAVI device, and a step of deploying a debris protection device in the aortic arch.

A second row 306 shows a step of inserting a TAVI guide wire and pigtail via the left femoral artery and a pacer via the venous access, as steps in the TAVI procedure, and notes that the debris protection device is deployed.

A third row 310 shows a step of obtaining right femoral arterial access and inserting a guide wire, as steps in the TAVI procedure, and notes that the debris protection device is deployed with an example pore size of 120 microns, while noting that the potential debris source can be aortic wall debris from deploying the tools in the aorta.

A fourth row 314 shows a step of a guide wire reaching the left ventricle, as a step in the TAVI procedure, notes that the debris protection device is deployed with an example pore size of 120 microns, and notes that the potential debris source can be aortic wall debris and also aortic valve debris.

A fifth row 318 shows a step of insertion of a TAVI prosthesis delivery system, notes that the debris protection device is deployed with an example pore size of 120 microns, and notes that the potential debris source can be aortic wall debris.

A sixth row 322 shows a step of positioning of the TAVI prosthesis delivery system, notes that the debris protection device may optionally now be activate to a smaller pore size, for example a pore size of 30 microns and optionally a debris capture device may also be activated to capture debris, and notes that the potential debris source can be aortic wall debris.

A seventh row 326 shows a step of accelerating the heart rate, called pace-up, notes that the debris protection device may optionally now be at a smaller pore size of 30 microns and a debris capture device may optionally be activated, and notes that the potential debris source can be aortic wall debris.

An eighth row 330 shows a step of deploying the TAVI prosthesis, notes that the debris protection device may optionally now be at a smaller pore size of 30 microns and a debris capture device may optionally be activated, and notes that the potential debris source can be from overriding the aortic valve with the prosthesis.

A ninth row 334 shows a step of pace-down, after stopping the rapid pacing, notes that the debris protection device may optionally now be at a smaller pore size of 30 microns and a debris capture device may optionally be activated, and notes that the potential debris source is now potentially what is called a debris shower.

A tenth row 338 shows an optional step of expanding a TAVI balloon, if needed, notes that the debris protection device may optionally now be at a smaller pore size of 30 microns and a debris capture device may optionally be activated, and notes that the potential debris source is now potentially what is called a debris shower.

An eleventh row 342 shows a step of retrieving the TAVI delivery system from the patient's body, at which time the debris protection device is optionally open, allowing the TAVI delivery system to pass, and notes that the potential debris source is now potentially the aortic wall.

A twelfth row 346 shows a step of closing the arterial and the venous access, at which time the debris protection device is optionally also retrieved from the patient's body.

It is expected that during the life of a patent maturing from this application many relevant debris protection devices will be developed and the scope of the term debris protection device is intended to include all such new technologies a priori.

As used herein with reference to quantity or value, the terms “about” and “approximately” mean “within ±25 % of”.

The terms “comprising”, “including”, “having” and their conjugates mean “including but not limited to”.

The term “consisting of”is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional”features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of managing debris-protection associated with medical procedures, the method comprising:
activating a debris-protection device placed in a body lumen to prevent debris from flowing with body fluids to a body organ prior to beginning a step in a medical procedure which is likely to produce debris; and
de-activating the debris-protection device before the activated debris-protection device is likely to cause damage to the body organ.

2. The method according to claim 1, wherein the activating comprises activating a debris capture device placed in an aorta.

3. The method according to claim 1, wherein the activating comprises activating a filtering of blood entering coronary arteries.

4. The method according to claim 1, wherein the activating comprises blocking blood from entering coronary arteries.

5. The method according to claim 1, wherein the activating is performed prior to performing a pace-up step in a valve replacement medical procedure.

6. The method according to claim 1, wherein the activating is performed simultaneously to performing a pace-up step in a valve replacement medical procedure.

7. The method according to claim 1, wherein the activating comprises activating filtering blood through a mesh with a mesh pore size in a range from 120 to 500 microns.

8. The method according to claim 1, wherein the activating comprises activating prior to positioning an implantable device at a location at a heart.

9. The method according to claim 1, wherein the activating comprises activating filtering blood through a mesh with a mesh pore size in a range from 30 to 120 microns.

10. The method according to claim 1, wherein the activating comprises activating filtering blood through a mesh with a mesh pore size in a range from 0 to 6 microns.

11. The method according to claim 1, wherein the activating comprises activating prior to expanding an implantable device at a location at a heart.

12. The method according to claim 1, wherein the activating is performed prior to performing a pace-down step in a valve replacement medical procedure.

13. The method according to claim 1, wherein de-activating is performed after a duration in a range from 5 seconds to 60 seconds.

14. The method according to claim 1, wherein de-activating is performed after a duration in a range from 5 seconds to 120 seconds.

15. The method according to claim 1, wherein the de-activating is performed automatically by a timer.

16. The method according to claim 1, wherein the de-activating is performed automatically based on a signal from a sensor.

17. The method according to claim 1, wherein the activating is performed automatically based on a signal from a sensor.

18. The method according to claim 1, wherein the activating comprises activating a debris capture device placed in a Vena Cava.

19. The method according to claim 1, wherein the activating comprises activating a debris filtering device placed in a Vena Cava.

20. The method according to claim 1, wherein the activating requires exerting force.

21-27. (canceled)
